(19) United States
(12) Patent Application Publication
(10) Pub. No.: US 2004/0086136 A1
(43) Pub. Date: May 6, 2004

(54) Electro-aero-acoustic source and system for active noise control

(76) Inventors: Jean-Laurent Peube, Naintre (FR); Christian Carme, Marseille (FR)

Correspondence Address: Oliff & Berridge, PO Box 19928, Alexandria, VA, 22320, US

(21) Appl. No.: 10/275,507

(22) PCT Filed: May 10, 2001
(86) PCT No.: PCT/FR01/01414

(30) Foreign Application Priority Data
May 11, 2000 (FR) 0006018

(51) Int. Cl. A61F 11/06; G10K 11/16; B06B 1/20

(52) U.S. Cl. 381/71.5

(57) ABSTRACT

The invention relates to an electroaeroacoustic source (2) placed in a flow (V) or at the edge of a flow, consisting of an electrodynamic or electromagnetic motor means (5) generating an oscillating motion, and an element (3) coupled to the motor means (5) obstructing the flow so as to exert a dynamic action on said flow. This source can be used to produce an electroaeroacoustic system for the active noise control, particularly in a confined flow.

The present invention relates to an electroaeroacoustic source as well as an electroaeroacoustic system applying such a source, said electroaeroacoustic source and system being intended in particular for active noise control in a confined flow.

The pipes connecting reciprocating machines such as compressors and heat engines, or the outputs of rotating turbine-driven or screw-driven devices such as fans and pumps, give rise to fluctuations in flow characteristics which bring about electroaeroacoustic emissions that in turn cause noise nuisance. The latter may be attenuated passively by devices known as “mufflers,” but because of their necessarily limited size they are often ineffective for low-frequency sounds.

Noise emitted by the aforementioned fluctuations can also be attenuated by the now well-known active absorption procedures using loudspeakers. However, the sound energy requirement is very high so that the loudspeakers must be very large and are consequently difficult to implement. Moreover, since loudspeakers are light, movable mechanical structures, they do not have the proper acoustic impedance to act in a fluid channel, which may also be the site of hot or corrosive flow as in the case of discharge pipes from heat engines or refrigeration compressors.

The goal of the present invention is to overcome these drawbacks by providing a particularly effective device for eliminating sound nuisances that is advantageously able to replace a loudspeaker under conditions whether the latter cannot be used for the reasons stated above, particularly inside pipes conducting a flow.

For this purpose, the invention essentially relates to an electroaeroacoustic source comprised of an electrodynamic or electromagnetic motor means able to generate an oscillating movement, and at least one element coupled to said motor means in such a way as to execute an oscillating movement, and locatable in a flow or at the edge of a flow, and forming an obstacle to the flow so that a dynamic action is exerted on this flow.

The device according to the invention, known as an electroaeroacoustic source and defined above, is based on the following theoretical principle:

Emission of an acoustic wave in the case of a movable wall results from the stresses exerted on the wall by the fluid, whereby the pressure fluctuations impacting the wall constitute an acoustic radiation source. A flow exerts lift and drag forces on an obstacle that are proportional to the square of the velocity. If a vibratory translational or rotational movement, or a combination of these two types of movements, is applied to this obstacle by an appropriate electrodynamic or electromagnetic means, an acoustic source associated with a variation of these forces as a function of time is created. The acoustic emission thus obtained has the same spectral content as the fluctuation of the above forces.

The electroaeroacoustic source according to the invention, based on the general principles outlined above, comprises an element of appropriate shape forming an obstacle to which an oscillating movement is applied, exerting a dynamic effect on the flow in which it is placed. The oscillating movement is appropriately controlled by a means such as a linear or rotary electrical motor, preferably oscillating, or by an oscillating element in a magnetic induction field, preferably an oscillating coil, this means being itself controlled by an electrical signal, particularly in the application to provision of an active noise control system.

The electroaeroacoustic sources according to the invention may have high power levels due to the flow-amplifying effect for the fluctuating force exerted by the fluid on the wall of the element forming an obstacle.

In order to establish the center position of this obstacle-forming element, particularly in the case of such an organ describing an oscillating movement about an axis, means are advantageously provided such as a helical or leaf spring to create an elastic spring force urging said element to its center position. Adjusting means can be provided to modify the center position of the obstacle-forming element, so that its acoustic emission characteristics can be modified.

According to one particular embodiment, these elastic spring means also support the obstacle-forming element by defining a “virtual” axis of rotation. This arrangement is particularly advantageous in the case where mechanical rotating bearings such as ball bearings must be avoided; this is the case in particular when the electroaeroacoustic source must operate in a hot and/or corrosive flow such as refrigeration compressor fluids or combustion gases.

The electroaeroacoustic source according to the invention has different directivity properties depending on the aerodynamic characteristics of the obstacle-forming element, under the conditions in which this element is placed. The directivity of the acoustic emission from such a source is established by the direction of the force exerted by the obstacle-forming element on the fluid.

An obstacle-forming element, which may be shaped or not shaped, mounted so that it oscillates about an axis transverse to the direction of flow, and creating a wake, has a directivity that is principally oriented parallel to the direction of flow, since said obstacle-forming element has no lift.

One example of an obstacle-forming element meeting the above definition is a substantially rectangular oscillating flap whose oscillation axis preferably passes through the center of the flap.

Another example of an obstacle-forming element that still has directivity parallel to the direction of flow is an oscillating flap having curved walls on its leading edge and its trailing edge, said walls preventing the flow from re-adhering to the wall of the flap and thus ensuring that the wake width is proportional to the angle of attack, such as an “S”-shaped flap; this variant of the obstacle-forming element is easier to produce industrially.

The electroaeroacoustic sources defined above, whose directivity is principally parallel to the direction of flow may be termed “axial” electroaeroacoustic sources.

It is also possible to make electroaeroacoustic sources according to the invention whose directivity is substantially perpendicular to the direction of flow, and which can be termed “transverse” electroaeroacoustic sources. Contrary to axial sources, a transverse electroaeroacoustic source must have an obstacle-forming element those lift is large relative to the flow. A typical example of such an element is an oscillating flap with a wing shape.

Since the above-defined electroaeroacoustic sources are single sources, they can be multiplied to create a multiple electroaeroacoustic source combining two or more similar elementary electroaeroacoustic sources, all of the axial type or the transverse type, with all these sources being disposed “in parallel” or “in series” in the flow or in a combination of these arrangements, and they are given the same oscillating movement.

From the electroaeroacoustic sources defined above, it is also possible to create complex electroaeroacoustic sources enabling varied and specific effects to be obtained from the interaction between neighboring fixed or mobile obstacles and from the different values of the moments of inertia and aerodynamic forces, as a function of the dimensions of the obstacles. Complex electroaeroacoustic sources can in particular be obtained:

by combining a fixed obstacle with at least one axial or transverse electroaeroacoustic source (as defined above);

by combining at least one axial electroaeroacoustic source with at least one transverse electroaeroacoustic source such that the main direction of the emission, which is the resulting force of the lift and drag forces of the two sources, is an oblique direction relative to the direction of flow.

The electroaeroacoustic sources defined above were defined relative to an indefinite flow, disregarding its direction; this flow could be either free or confined by walls, for example the walls of a pipe. Confining the flow by walls offers the advantageous option of increasing the efficiency of the electroaeroacoustic sources defined above by disposing such a (single or multiple) source next to an appropriate nozzle that creates a local narrowing in the cross section of the pipe and hence of the flow, thus accelerating the flow and increasing the force exerted on this flow by the electroaeroacoustic source. Such a nozzle may be formed by a simple fixed, shaped sleeve inserted into the pipe traversed by the flow, with the electroaeroacoustic source being disposed inside the sleeve in the reduced cross section delimited by this sleeve.

In one variant, the nozzle or sleeve that reduces the cross section available for flow is created by movable walls, particularly walls articulated on an axis, associated with mechanical control means; the fixed but adjustable position of these walls enables the cross section available for passage of the flow to be modified and hence the velocity to be increased or decreased where the electroaeroacoustic source is located, so that the efficiency of this source can be controlled as desired.

According to another variant, the electroaeroacoustic source, located at the edge of the flow, has an obstacle-forming element comprised of part of the oscillating wall confining the flow, whereby this movable wall part also narrows the flow cross section. In this embodiment, the oscillating obstacle-forming element is located at the edge of the flow, and itself creates the nozzle.

All the electroaeroacoustic sources defined above can be implemented with a view to active noise control, also known as active acoustic absorption, particularly in a confined flow with the configurations (nozzles) described above. In the context of this preferred application, the invention provides an electroaeroacoustic system for active noise control that comprises essentially at least one electroaeroacoustic source as defined above, with an electrodynamic or electromagnetic motor and an obstacle-forming element, located in a flow or at the edge of a flow, as well as a controller to control said motor means, the controller being itself controlled by at least one microphone that measures the noise to be controlled.

In the case of such an electroaeroacoustic system for active noise control using a single electroaeroacoustic source, or a plurality of identical elementary sources fed in parallel, the controller can be a multi-channel controller. In the case of a system using two or more electroaeroacoustic sources with different characteristics or types, for example one axial source and one transverse source (as defined above), powered separately, the controller will be a multichannel controller.

The electroaeroacoustic systems for active noise control according to the invention may use only the electroaeroacoustic sources as defined above as active noise control means, in which case they are known as “pure” electroaeroacoustic systems. “Combined” systems are also possible, using at least one electroaeroacoustic source as defined above plus active or passive noise attenuation means operating on a different principle.

Thus, a combined system may include at least one electroaeroacoustic source plus a loudspeaker; the loudspeaker handles high frequencies, which electroaeroacoustic sources emit only with difficulty. The loudspeaker can be replaced by another active means such as a controlled electropneumatic source.

For the passive means associated with at least one electroaeroacoustic source, particularly a source placed in a nozzle (as defined above), it is advantageous for the sleeve to be a nozzle made of absorbant acoustic material. Such a combination forms a “semi-active” system for noise control in a confined flow, in which system the electroaeroacoustic source(s) handle(s) the low and medium frequencies while the high frequencies are absorbed passively by the aforesaid sleeve made of an appropriate material.

In any event, the invention will be better understood with the aid of the description hereinbelow referring to the attached schematic drawings that show embodiments of these electroaeroacoustic sources, and electroaeroacoustic systems for active noise control using such sources, as examples:

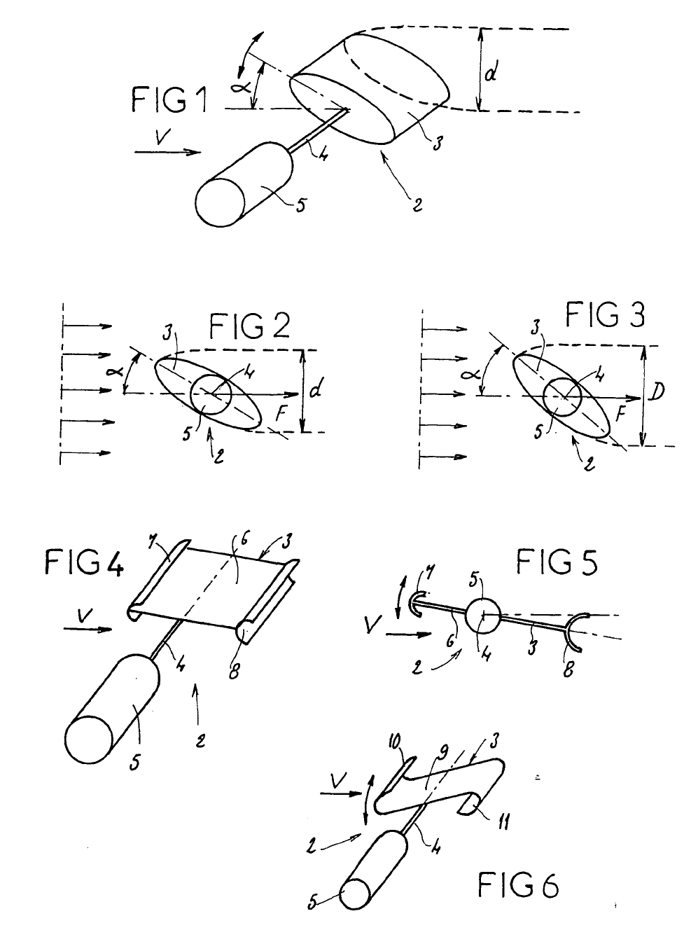

FIG. 1 is a general perspective diagram of an electroaeroacoustic source according to the invention;

FIG. 2 is a side view of the source in FIG. 1, located in a free flow and constituting an axial source;

FIG. 3 is a view similar to that of FIG. 2, but illustrating another arrangement of this axial source;

FIG. 4 is a perspective view of an electroaeroacoustic source according to the invention, with a particular shape;

FIG. 5 is a side view of the source in FIG. 4;

FIG. 6 is a perspective view of another electroaeroacoustic source according to the invention, with a particular shape;

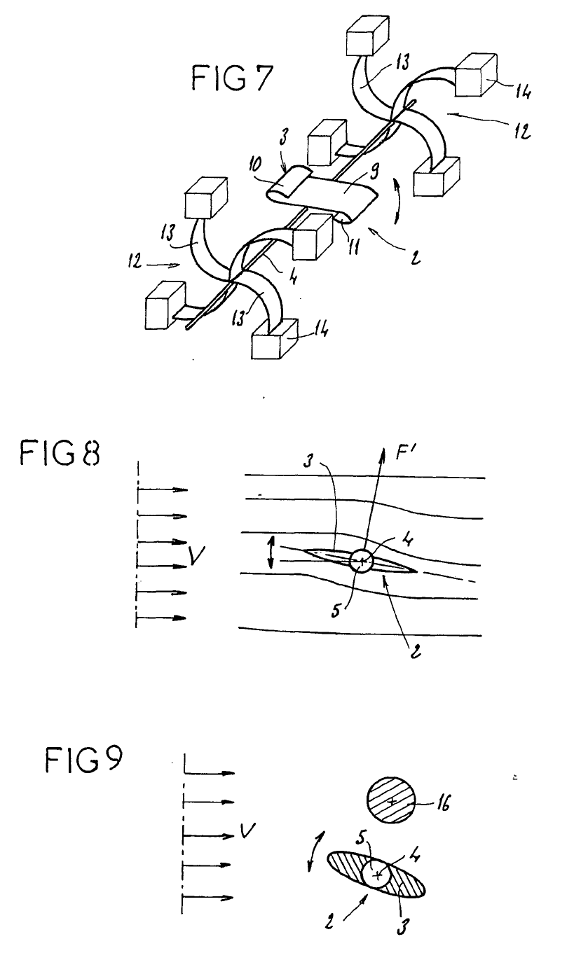

FIG. 7 is a perspective view illustrating a particular installation of an electroaeroacoustic source such as that in FIG. 6;

FIG. 8 is a side view of an electroaeroacoustic source such as that in FIG. 1, located in a flow and constituting a transverse source;

FIG. 9 is a general diagram of a complex electroaeroacoustic source;

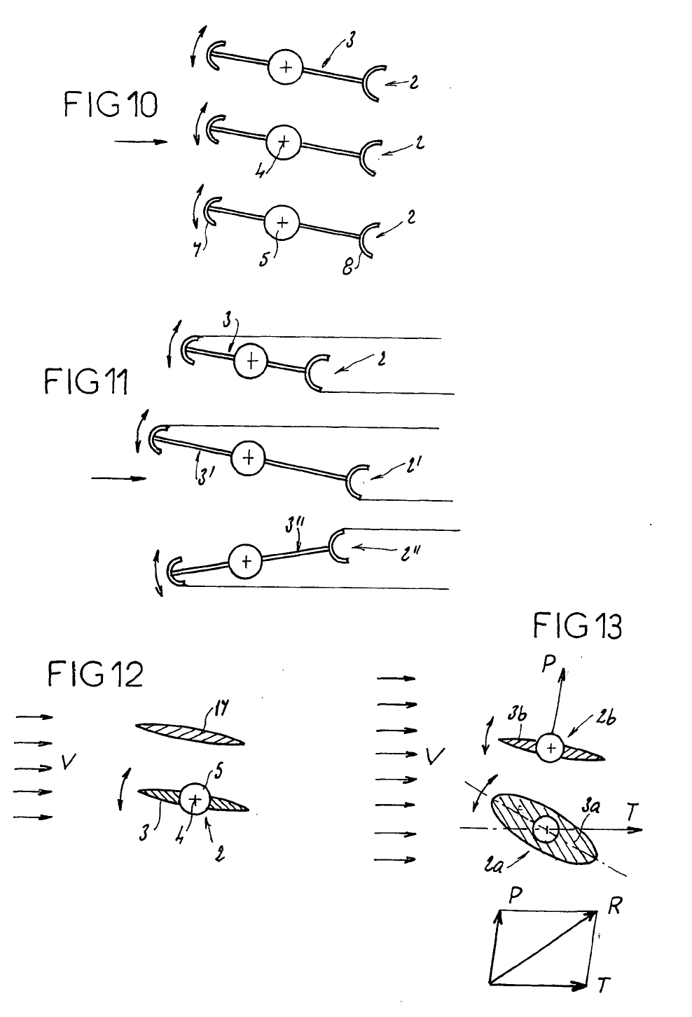

FIG. 10 is a general view of a multiple electroaeroacoustic source;

FIG. 11 is a general view of another multiple electroaeroacoustic source;

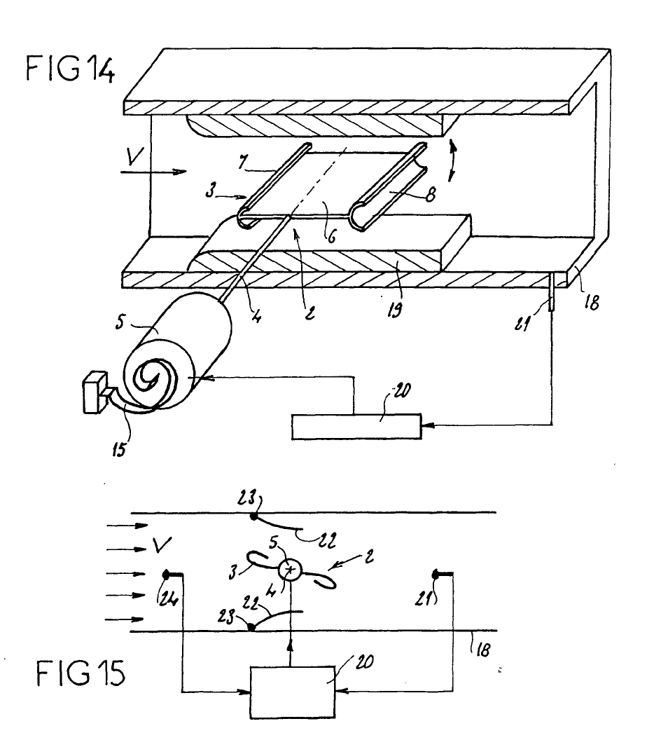

FIGS. 12 and 13 illustrate examples of specific complex sources;

FIG. 14 shows, in perspective and in cross section, an electroaeroacoustic source located in a confined flow;

FIG. 15 is a general diagram of another source located in a confined flow, and usable for active noise control;

FIGS. 16, 17, 18, 19, 20, and 21 represent, in the form of general diagrams, other examples of sources used for active noise control.

As shown in FIG. 1, an electroaeroacoustic source, designated overall by 2, includes a movable obstacle 3, mounted on the axis 4 of an electrodynamic or electromagnetic motor element 5 such as an oscillating electric motor or coil oscillating in a magnetic induction field, supplied by an electric current, which is able to vary the angle α of obstacle 3 in an oscillating manner.

Such a device is located in a fluid flow with a velocity V, and the obstacle 3, which moves in oscillating fashion, then exerts a dynamic action on the flow.

FIG. 2 shows an electroaeroacoustic source 2 as described above, located in a free flow with a velocity V, with obstacle 3 forming an average angle α with the direction of this flow, thus creating a wake with a transverse dimension d. In the case of FIG. 3, the same obstacle 3 forms a larger angle α and creates a wake with a larger transverse dimension D. The drag force exerted by the flow with velocity V on obstacle 3 is greater in the case of FIG. 3 than in that of FIG. 2, while the lift force is largely unchanged, for incidences where the flow is highly separated. The force, with an oscillating nature, exerted by obstacle 3 on the fluid is marked F. The directivity of the acoustic emission of electroaeroacoustic source 2 is established by the direction of this force F; this directivity is principally oriented parallel to the flow direction; in this case it is an “axial” source.

Since obstacle 3 must essentially create a wake, without a significant lift effect, to obtain such an axial source, FIGS. 4 to 7 illustrate examples of specially shaped sources meeting these requirements.

According to FIGS. 4 and 5, obstacle 3 is in the shape of an oscillating rectangular flap that has a first curved wall 7 on its leading edge and a second curved wall 8 on its trailing edge, with the axis 4 of motor 5 passing through the center of flap 6. The curved walls 7 and 8 prevent the flow from re-adhering to the wall of flap 6, thus ensuring that the width of the wake is proportional to angle of incidence α.

According to FIG. 6, obstacle 3 is comprised of a rectangular flap 9 whose leading and trailing edges are curled in opposite directions, as indicated at 10 and 11, in order to give flap 9 an S shape. Curled edges 10 and 11 thus constitute curved walls ensuring that the desired wake is produced; moreover, this shape facilitates industrial manufacture of obstacle 3.

FIG. 7 illustrates a particular installation of electroaeroacoustic source 2, like that in FIG. 6, with an S-shaped flap 9. Axis 4, which is equated with the axis of symmetry of flap 9, is supported by two bearings 12, each composed of several leaf springs 13 attached to a fixed part 14, with the leaf springs 13 serving as supports for axis 4, hence obstacle 3, and elastic means for urging obstacle 3 back into its center position relative to which it oscillates and produces a sound emission that interacts with the flow.

The spring function that returns shaped obstacle 3 to its center position can also be accomplished by a simple helical spring 15, this variant being illustrated in FIG. 14 (described in detail below).

FIG. 8 shows another electroaeroacoustic source 2 which, contrary to the preceding sources, is a “transverse” source, namely its directivity is substantially perpendicular to the flow direction, with velocity V. For this purpose, source 2 has a wing-shaped obstacle 3, still oscillating under the action of a motor 5, whereby such an obstacle 3 creates a force F in the fluid, principally due to lift.

FIG. 9 illustrates a first example of an electroaeroacoustic source known as a “complex” source, composed of an axial source 2 with an obstacle 3 oscillating under the action of a motor 5, as described above, which is located in the vicinity of a fixed obstacle 16. The movable obstacle 3 and fixed obstacle 16 are placed in a fluid flow with velocity V. The instantaneous increase in the incidence of movable obstacle 3 brings about an increase in the fluid velocity past fixed obstacle 16 due to deflection of the flow, which has the effect of increasing the pressure on this obstacle 16, in its instantaneous aeroacoustic emission. The two obstacles 3 and 16, one of which is oscillating and powered and the other is fixed, thus constitute an electroaeroacoustic source with a specific structure and function, whose efficiency is greater than that of source 2 alone, which in this case is an axial source.

FIG. 10 shows an electroaeroacoustic source known as a “multiple” source, which is composed of several single sources 2 as described above. In this example, there are three axial sources 2, mounted “in parallel” in the same fluid flow, with each source 2 having an oscillating flap 6 driven by a motor 5. The three sources 2 have identical shapes and dimensions in this case, and are given the same oscillating movement. It will be noted that replacement of a single electroaeroacoustic source, which varies the wake width of a given amplitude, by synchronous electroaeroacoustic sources in parallel, whose size is reduced in proportion to the number n, gives a torque that is n³ times smaller for each source and, in total an electromechanical power that is n times smaller. In addition, the sound emission of sources 2 thus grouped together is reinforced by their interaction effect, which is similar to that described previously for the combination of a source 2 with an oscillating obstacle 3 and a fixed obstacle 16 (FIG. 9).

As shown in FIG. 11, it is also possible to group several single electroaeroacoustic sources 2, 2′, and 2″ with different characteristics, particularly with different dimensions and adapted to pulses and amplitudes with different frequency bands, whereby such a complex source enables various effects to be achieved, reinforced by the interaction of the flows around the respective obstacles 3, 3′, and 3″. The lowest frequencies are made for example with a broader source than the medium frequencies, and a much broader source than the lowest frequencies. In particular, FIG. 1 shows an element forming a complex electroaeroacoustic source design where the lowest pulse is emitted by source 2′ located in the center, the medium pulse is mainly emitted by the lower source 2″, while the high pulse is produced by the upper source 2.

FIG. 12 shows another complex electroaeroacoustic source design composed of a transverse source 2 with an obstacle 3 oscillating under the action of a motor 5, which is located in the vicinity of a fixed wing-shaped obstacle 17 similar to the shape of movable obstacle 3. The reinforcing effect of fixed obstacle 17 is similar, in this case, to that of fixed obstacle 16 associated with an axial source in the example of FIG. 9.

FIG. 13 shows still another complex electroaeroacoustic source design which combines an axial source 2a and a transverse source 2b such that the main emission direction is the oblique direction of the resultant force R of the impermanent drag force T of axial source 2a and lift force P of transverse source 2b. Here as well, the acoustic emission is reinforced because of the interaction of the two respective obstacles 3a and 3b and the two sources 2a and 2b located in the same flow with velocity V.

In all the examples described thus far, the electroaeroacoustic sources are assumed to be located in a free flow with an indefinite transverse dimension. FIGS. 14 and following, however, represent electroaeroacoustic sources located in a confined flow, i.e. in practice in a pipe 18 traversed by a fluid, whose velocity is always indicated as V. These sources are applied to active noise control.

FIG. 14 shows a single electroaeroacoustic source 2, designed as described above, with an oscillating obstacle 3 located inside pipe 18 traversed by the flow. In particular, the oscillating obstacle 3 of this source 2 is located in a nozzle 19, accommodated and fixed in pipe 18, which causes a local reduction in the cross section of pipe 18, thus speeding up fluid flow and increasing the force applied by this source 2 to this flow.

An electroaeroacoustic system for active noise control is made from this configuration by providing a controller 20 controlling the motor 5 of source 2 by means of a microphone 21 measuring the noise to be controlled, for example at the wall of pipe 18.

FIGS. 15 and following illustrate various embodiments of sources located in a confined flow and applied to active noise control according to the principle described above.

FIG. 15 shows an electroaeroacoustic source 2 located in a pipe 18 near a nozzle made of walls 22 each articulated on an axis 23. In this case as well, the nozzle reduces the cross section available for flow, and in the present case an appropriate mechanical control device (not shown) pivots movable walls 22 and adjusts their position in order to adjust the cross section available for the flow. Moving walls 22 closer together and obstructing the cross section increases the efficiency of source 2 for a given oscillation amplitude of its flap 9. It may be useful to retract movable walls 22 when it is no longer desirable to place electroaeroacoustic source 2 in operation.

In addition, FIG. 15 shows the active noise control system with its single-channel controller 20, in relation with motor 5 of source 2, with a control microphone 21, and also with a reference microphone 24, the two microphones 21 and 24 being placed inside the pipe 18, downstream and upstream respectively of source 2.

FIG. 16, in which the elements corresponding to those previously described have the same reference numerals, shows a multiple electroaeroacoustic source comprising two identical single sources 2 disposed in parallel. The multiple source thus formed is located in pipe 18, near a nozzle 19. The single-channel controller 20 in this case powers the motors 5 of the two single sources 2 synchronously.

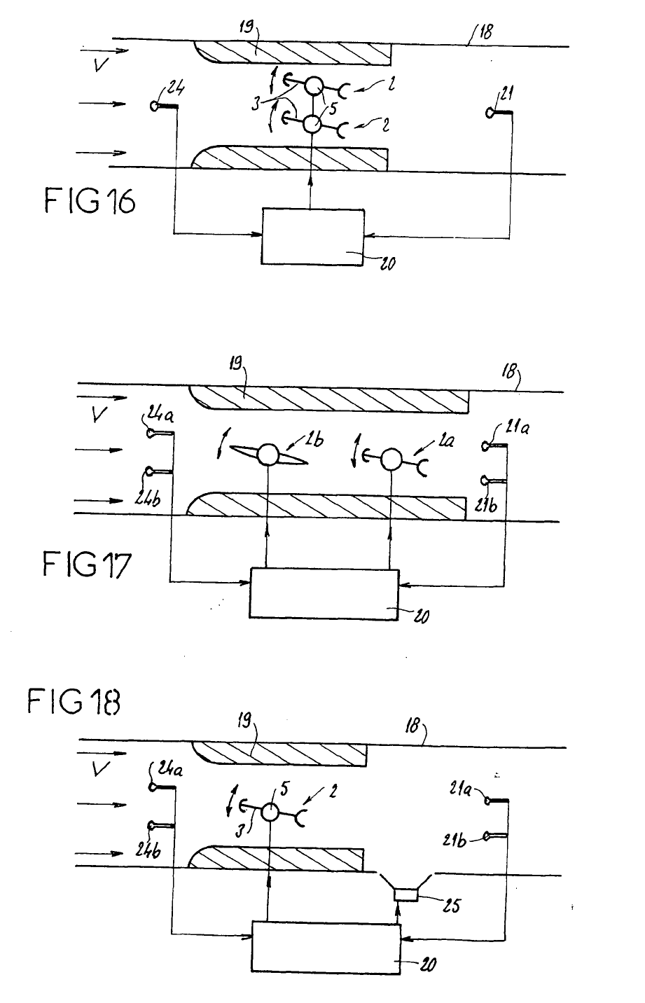

FIG. 17 shows an active noise control system using a complex source resulting from two single electroaeroacoustic sources 2a and 2b, that are axial and transverse respectively, disposed in series inside pipe 18, near nozzle 19. The two sources 2a and 2b are powered separately by a multichannel controller 20 receiving signals from two control microphones 21a and 21b and two reference microphones 24a and 24b.

As shown in FIG. 18, the active noise control system can employ an activator operating on a different principle, particularly a loudspeaker 25. The electroaeroacoustic source 2 is located in pipe 18, near nozzle 19. Loudspeaker 25 is located on the wall of pipe 18. A multichannel controller 20 separately powers the motor 5 of source 2 and the loudspeaker 25. The latter handles high frequencies, which it is difficult for electroaeroacoustic source 2 to emit. This active noise control system can be applied in particular to ventilation devices, where the normally non-corrosive environment enables loudspeakers to be used.

In the examples of FIGS. 14, 16, 17, and 18, nozzle 19 has a generally cylindrical shape and results from a fixed, shaped sleeve inserted into pipe 18 traversed by the flow, this sleeve being solid or at least solid-walled.

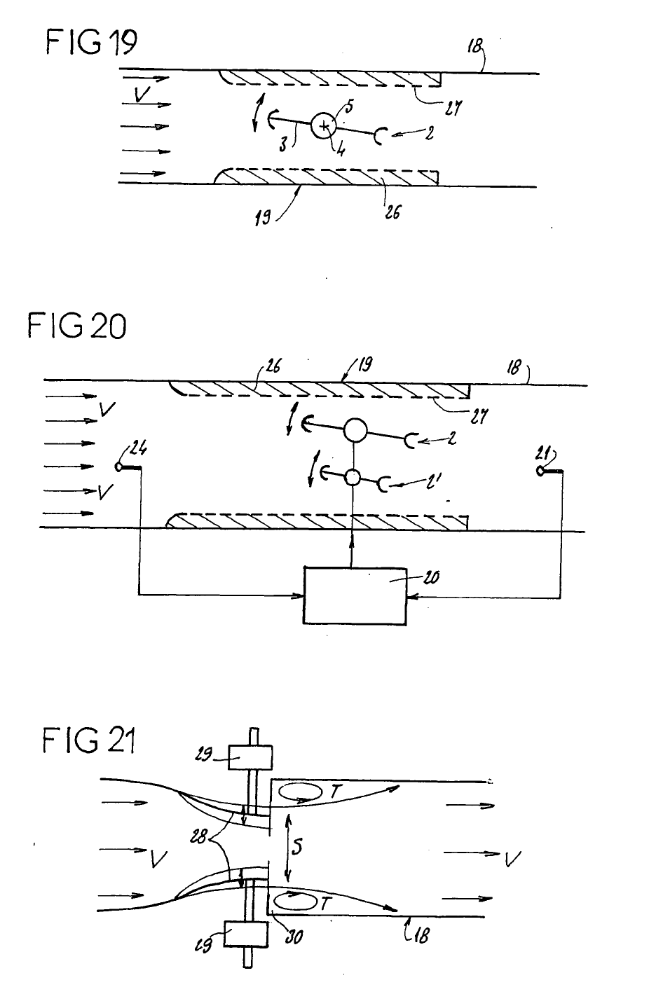

FIGS. 19 and 20 illustrate variants in which the nozzle 19 is comprised of a fixed, shaped sleeve made of an absorbant acoustic material 26 that may be contained in a perforated-wall envelope 27. In the configuration of FIG. 19, the electroaeroacoustic source 2, which is a single source, handles the low frequencies while specially shaped nozzle 19 absorbs the high frequencies. In the configuration of FIG. 20, the low and medium frequencies are handled by the complex electroaeroacoustic source made of two single sources 2 and 2′ in parallel driven by single-channel controller 20, while the high frequencies are absorbed passively by the absorbant acoustic material 26 of nozzle 19. The assembly thus constitutes a semi-active system circuit control system in a confined flow. Sic; French circuit. It is possible that bruit (“noise”), was intended. This is on French page 13, line 17. Translator.

Finally, FIG. 21 shows another embodiment of the invention wherein pipe 18 has a part 28 that narrows the cross section available for flow, this part 28 being formed of flexible walls that can be made to vibrate by electromagnets 29 located outside pipe 18. In this case, the movable walls 28 play the role of the oscillating obstacle in the previous embodiments.

It will be noted that the configuration of FIG. 21 creates a pressure differential due to the existence of vortical wakes T in the sudden enlargement 30 of the cross section following the narrow portion resulting from the shape of movable walls 28. Indeed, as already explained above, the axial acoustic effect is practically zero if the obstacle is a shaped obstacle for which no flow separation is observed.

The following will not be a departure from the invention as defined in the attached claims:

in the case of a multiple electroaeroacoustic source, increasing or decreasing the number of single sources combined together;

in the case of an active noise control system, placing all forms of single or multiple or complex electroaeroacoustic sources in the pipe as described and illustrated in the drawings, using all appropriate arrangements of control microphones and, possibly, reference microphones.

1. Electroaeroacoustic source, characterized by being comprised of an electrodynamic or electromagnetic motor means (5) able to produce an oscillating movement, and at least one element (3) coupled to said motor means, in order to execute an oscillating movement, and locatable in a flow (V) or at the edge of a flow, forming an obstacle to the flow so as to exert a dynamic action on this flow.

2. Electroaeroacoustic source according to claim 1, characterized in that the motor means (5) is a rotary or linear electric motor, preferably oscillating.

3. Electroaeroacoustic source according to claim 1, characterized in that the motor means (5) is an element oscillating in a magnetic induction field, preferably an oscillating coil.

4. Electroaeroacoustic source according to any one of claims 1 to 3, characterized in that, to fix the center position of the element (3) forming an obstacle, such an element executing an oscillating movement around an axis (4), means such as a helical spring (15) or leaf spring (13) creating an elastic force returning said element (3) to its center position are provided.

5. Electroaeroacoustic source according to claim 4, characterized in that the elastic return means (13) also support element (3) forming an obstacle by defining a “virtual” axis of rotation (4).

6. Electroaeroacoustic source according to claim 4 or 5, characterized in that the means for regulating the center position of element (3) forming an obstacle are provided to modify its acoustic emission characteristics.

7. Electroaeroacoustic source according to any one of claims 1 to 6, characterized in that it has an element (3) forming an obstacle, in shaped or non-shaped form, mounted to oscillate about an axis (4) transverse to the direction of flow (V) and creating a wake, which has a directivity oriented mainly parallel to the flow direction (V) in order to constitute an axial electroaeroacoustic source (2).

8. Electroaeroacoustic source according to claim 7, characterized in that the element (3) forming an obstacle is a substantially rectangular oscillating flap (6), with the oscillation axis (4) preferably passing through the center of inertia of flap (6).

9. Electroaeroacoustic source according to claim 7, characterized in that the element (3) forming an obstacle is a flap (6; 9) that has curved walls (7, 8; 10, 11) on its leading edge and trailing edge such as an S-shaped oscillating flap (9).

10. Electroaeroacoustic source according to any one of claims 1 to 6, characterized in that it has an element (3) forming an obstacle that has a high lift force relative to the flow (V) such as a wing-shaped oscillating flap, and which thus has directivity substantially perpendicular to the direction of flow (V) in order to constitute a transverse electroaeroacoustic source (2).

11. Electroaeroacoustic source according to any one of claims 1 to 10, characterized by being designed as a complex electroaeroacoustic source combining a fixed obstacle (16; 17) with at least one axial or transverse electroaeroacoustic source (20).

12. Electroaeroacoustic source according to any one of claims 1 to 10, characterized by being designed as a multiple electroaeroacoustic source, combining two or more similar individual electroaeroacoustic sources (2) of the axial or transverse type, all these sources (2) being disposed in the flow (V) in parallel or in series, or in a combination of these arrangements, and having the same oscillating movement.

13. Electroaeroacoustic source according to any one of claims 1 to 10, characterized by being designed as a complex electroaeroacoustic source, combining at least one axial electroaeroacoustic source (2a) and at least one transverse electroaeroacoustic source (2b) such that the main emission direction (R) is oblique relative to the direction of flow (V).

14. Electroaeroacoustic source according to any one of claims 1 to 13, characterized by being located in a confined flow (V), particularly in a pipe (18).

15. Electroaeroacoustic source according to claim 14, characterized by being disposed near a nozzle (19) or a fixed, shaped sleeve inserted into a pipe (18) traversed by flow (V), to create a local narrowing in the cross section of this flow (V).

16. Electroaeroacoustic source according to claim 15, characterized in that the nozzle or sleeve is created by movable walls (22), particularly articulated about an axis (23), associated with mechanical control means, so that the cross section available for the passage of flow (V) can be adjusted.

17. Electroaeroacoustic source according to claim 14, characterized in that it is located at the edge of flow (V) and in that it has an obstacle-forming element comprised of part of oscillating wall (28) confining the flow (V), this part of movable wall (28) thus narrowing the cross section of flow (V).

18. Electroaeroacoustic system for active noise control in an open or confined space, characterized in that has at least one electroaeroacoustic source (2) according to any one of claims 1 to 17, with an electrodynamic or electromagnetic motor means (5) and an element (3) forming an obstacle, located in a flow (V) or at the edge of a flow, as well as a controller (20) controlling said motor means (5), the controller (20) itself being controlled from at least one microphone (21) measuring the noise to be controlled.

19. Electroaeroacoustic system for active noise control according to claim 18, characterized in that it uses a single electroaeroacoustic source (2) or a plurality of identical single sources (2) powered in parallel, the controller (20) being of the single-channel type.

20. Electroaeroacoustic system for active noise control according to claim 18, characterized in that it uses two or more electroaeroacoustic sources with different characteristics or types, for example an axial source (2a) and a transverse source (2b), powered separately, the controller (20) being of the multi-channel type.

21. Electroaeroacoustic system for active noise control according to any one of claims 18 to 20, characterized in that it uses both a single electroaeroacoustic source (2) and an active (25) or passive (26) means for noise attenuation operating on a different principle.

22. Electroaeroacoustic system for noise control according to claim 21, characterized in that it has at least one electroaeroacoustic source (20) and, as another active means, a loudspeaker (25).

23. Electroaeroacoustic system for active noise control according to claim 21, characterized in that it has at least one electroaeroacoustic source (2) located in a nozzle (19) according to claim 15 and, as a passive means, the sleeve constituting nozzle (19), made of absorbant acoustic material (26).
